(19) United States
(12) Patent Application Publication
(10) Pub. No.: US 2022/0416168 A1
(43) Pub. Date: Dec. 29, 2022

(54) PHOTOACTIVE MATERIALS

(71) Applicant: Sumitomo Chemical Co., Ltd, Tokyo (JP)

(72) Inventors: Kiran Kamtekar, Cambridgeshire (GB); Ben Gardner, Cambridgeshire (GB)

(73) Assignee: Sumitomo Chemical Co., Ltd, Tokyo (JP)

(21) Appl. No.: 17/771,455

(22) PCT Filed: Oct. 23, 2020
(86) PCT No.: PCT/GB2020/052686

§ 371 Date: Apr. 22, 2022

(30) Foreign Application Priority Data
Oct. 24, 2019 (GB) 1915461.6
Jul. 29, 2020 (GB) 2011796.6

(51) Int. Cl. H01L 51/00; C08G 61/12; C07D 495/14; G01N 21/3563; G01N 21/359

(52) U.S. Cl. CPC H01L 51/0036; C08G 61/126; H01L 51/0043; C07D 495/14; H01L 51/0071; G01N 21/3563; G01N 21/359; C08G 2261/124; C08G 2261/148; C08G 2261/146; C08G 2261/18; C08G 2261/228; C08G 2261/3246; C08G 2261/94; H01L 51/4253

(57) ABSTRACT

A material comprising a group of formula (I): (I) wherein: X and Y are each independently selected from S, O or Se; Ar1, Ar2, Ar3 and Ar4 are each independently an unsubstituted or a substituted benzene, an unsubstituted or a substituted 5- or 6-membered heteroaromatic group or are absent; A¹ and A² are each independently an unsubstituted or a substituted benzene, an unsubstituted or a substituted 5- or 6-membered heteroaromatic group, a non-aromatic 6-membered ring having ring atoms selected from C, N, S and O or are absent; R¹ is H or a substituent; R² and R³ are each independently H or a substituent; and * represents a point of attachment to a hydrogen or non-hydrogen substituent.

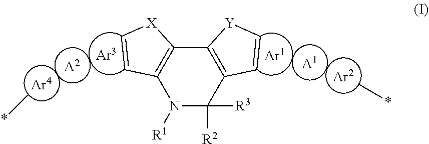

### BACKGROUND

Embodiments of the present disclosure relate to photoactive compounds and more specifically, but not by way of limitation, to photoactive compounds comprising electron donating groups.

Organic photovoltaic devices and organic photodetectors (OPDs) are known.

JP2015189853 relates to a polymer compound and an electronic element using the same.

WO 2017/155030 and WO 2019/054402 relate to tetrazolopyridine compounds.

WO 2017/078182 relates to benzimidazole fused heteroaryls.

WO 2012/146504 is directed to semiconductor materials based on dithienopyridone copolymers.

CN104211926 relates to a polymerization monomer for a donor material of a polymer solar battery.

KR2013070431 is directed to a multicyclic aromatic compound and organic light emitting device including the same.

US 2018/0175307 relates to organic electroluminescent materials and devices.

Pan et al., RSC Advances, volume 7, pages 3439-3442 is directed to a facilely synthesized lactam acceptor unit for high-performance polymer donors.

Cao et al., Dyes and Pigments, volume 139, pages 201-207 relates to D-A copolymers based on lactam acceptor unit and thiophene derivatives for efficient polymer solar cells.

### SUMMARY

A summary of aspects of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects and/or a combination of aspects that may not be set forth.

According to some embodiments, the present disclosure provides a material comprising an electron donor material.

The material may comprise a group of formula (I):

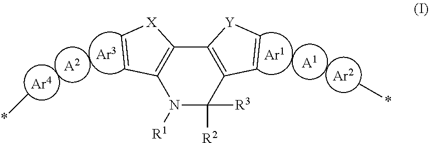

wherein:
X and Y are each independently selected from S, O or Se;
Ar¹, Ar², Ar³ and Ar⁴ are each independently an unsubstituted or a substituted benzene, an unsubstituted or a substituted 5- or 6-membered heteroaromatic group or are absent;
A¹ and A² are each independently an unsubstituted or a substituted benzene, an unsubstituted or a substituted 5- or 6-membered heteroaromatic group, a non-aromatic 6-membered ring having ring atoms selected from C, N, S and O or are absent;
R¹ is H or a substituent;
R² and R³ are each independently H or a substituent; and
* represents a point of attachment to a hydrogen or non-hydrogen group.

The group of formula I may have formula (Ia) or formula (Ib):

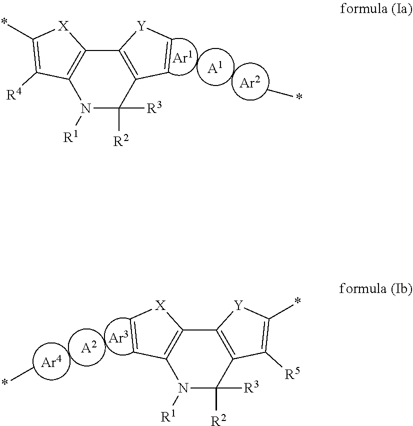

wherein:
X, Y, R¹, R² and R³ and * are as defined previously; and
R⁴ and R⁵ are each independently H or a substituent.

In some embodiments, Ar¹, A¹, Ar², Ar³ A² and Ar⁴ are absent and the group of formula (I) has formula (Ic):

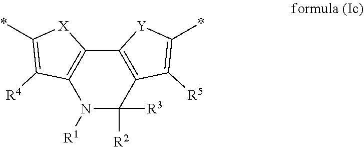

wherein:
X, Y, R¹, R², R³, R⁴, R⁵ and * are as defined previously.

In some embodiments, the material is a polymer comprising a repeat unit of formula (Id):

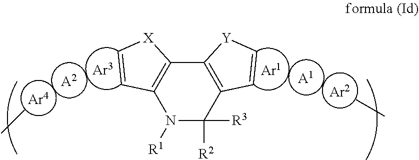

wherein X, Y, R¹ to R⁵, Ar¹ to Ar⁴, A¹ and A² are as previously defined previously.

Exemplary repeat units of formula (Id) are formulae (Ie), (If) and (Ig):

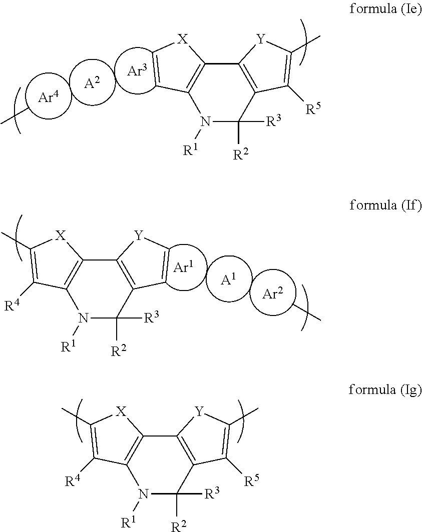

In some embodiments, the material comprises an electron accepting group, EAG.

In some embodiments, the compound comprising the group of formula (I) has formula (Ih), (Ii), (Ij) or formula (Ik):

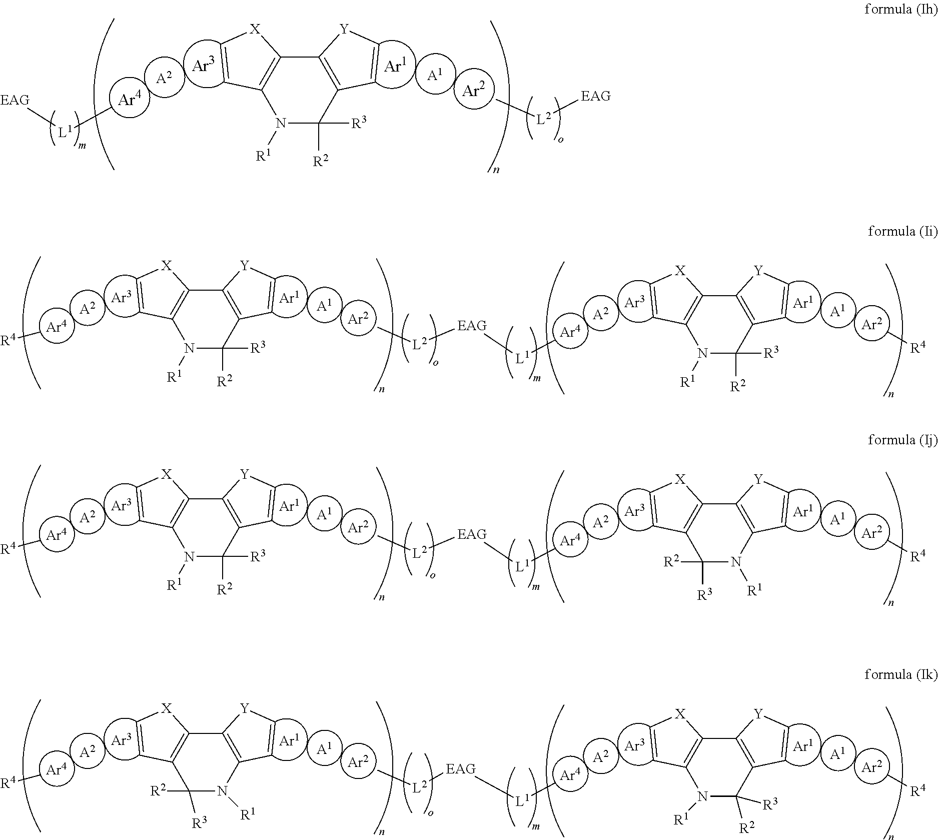

wherein:
n is an integer of 1 or more;
m and o are each independently 0 or an integer of 1 or more;
L¹ and L² each independently represent a bridging group when m and o are 1 or more or a direct bond when m and o are 0;
EAG represents an electron accepting group; and
X, Y, R¹ to R⁴, Ar¹ to Ar⁴, A¹ and A² are as previously defined previously.

In some embodiments, L¹ and L² are each independently a group of formula (II) or formula (III):

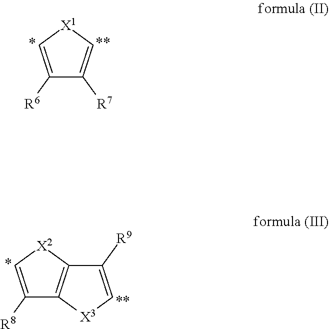

wherein:
X¹, X² and X³ are each independently S, O or Se;
* represents a point of attachment to Formula (Ih), Formula (Ii) or Formula (Ij);
** represents a point of attachment to EAG; and
R⁶, R⁷, R⁸ and R⁹ are each independently H or a substituent.

In some embodiments, each EAG is a group of formula (Via):

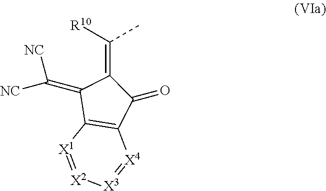

wherein:
R¹⁰ in each occurrence is H or a substituent selected from the group consisting of: C₁₋₁₂ alkyl wherein one or more non-adjacent, non-terminal C atoms may be replaced with O, S, COO or CO and one or more H atoms of the alkyl may be replaced with F; and an aromatic group Ar² which is unsubstituted or substituted with one or more substituents selected from F and C₁₋₁₂ alkyl wherein one or more non-adjacent, non-terminal C atoms may be replaced with O, S, COO or CO;
---- represents a linking position to Formula (I), L¹ or L²; and each X¹-X⁴ is independently CR¹² or N wherein R¹² in each occurrence is H or a substituent selected from C₁₋₂₀ hydrocarbyl and an electron withdrawing group.

Optionally, at least one R¹² is an electron-withdrawing group selected from F, Br, Cl and CN.

According to some embodiments there is provided a composition comprising an electron donor and an electron acceptor wherein at least one of the electron donor and electron acceptor is a material comprising a group of formula (I).

According to some embodiments, the material or composition as described herein is dissolved or dispersed in one or more solvents.

According to some embodiments, the present disclosure provides a photoresponsive device comprising an anode, a cathode and a photosensitive layer disposed between the anode and the cathode, wherein the photosensitive layer comprises a material as previously described.

The photoresponsive device may be an organic photodetector.

According to some embodiments, the present disclosure provides a photosensor comprising a light source and a photoresponsive device as described previously, wherein the photosensor is configured to detect light emitted from the light source.

According to some embodiments, the present disclosure provides a method of forming the organic photoresponsive device described previously comprising formation of the photosensitive organic layer over one of the anode and cathode and formation of the other of the anode and cathode over the photosensitive organic layer.

In some embodiments, formation of the photosensitive organic layer comprises deposition of a formulation comprising a composition as described herein dissolved or dispersed in one or more solvents.

In some embodiments, the light source emits light having a peak wavelength greater than 750 nm.

In some embodiments, the photosensor is configured to receive a sample in a light path between the organic photodetector and the light source.

According to some embodiments, the present disclosure provides a method of determining the presence and/or concentration of a target material in a sample, the method comprising illuminating the sample and measuring a response of a photoresponsive device as described previously.

### DESCRIPTION OF DRAWINGS

The disclosed technology and accompanying figures describe some implementations of the disclosed technology.

FIG. 1 illustrates an organic photoresponsive device according to some embodiments;

and

FIG. 2 shows absorption spectra for a polymer according to some embodiments of the present disclosure and a comparative polymer.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology.

Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that materials comprising a group of formula (I) may be used in a donor-acceptor system used in an organic photoresponsive device, e.g. a photovoltaic device such as a solar cell or an organic photodetector.

The materials may absorb long wavelengths of light, e.g. greater than about 750 nm, making them suitable for use in organic photodetectors for detection of light in the near-infrared range such as in the range of greater than about 750 nm, greater than 850 nm or greater than about 1000 nm. The materials may absorb wavelengths of light that are between about 750 nm and about 2000 nm, between about 750 nm and about 1000 nm or between about 1000 nm to about 2000 nm.

Absorption peaks of a material as described herein are as measured from a film of the material using a Perkin Elmer Cary 5000 UV-vis absorption spectrometer

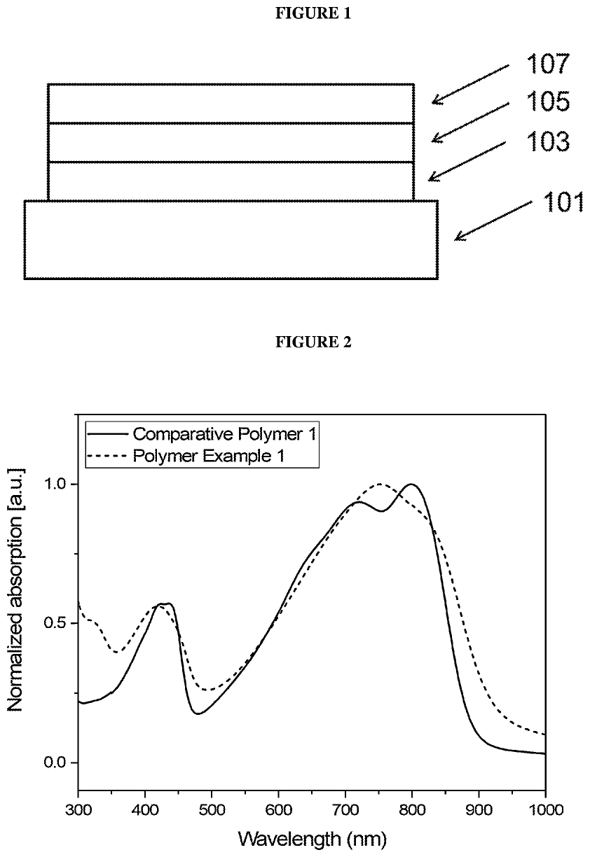

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g. due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor.

FIG. 1 illustrates an arrangement in which the cathode is disposed between the substrate and the anode. In other embodiments, the anode may be disposed between the cathode and the substrate.

The bulk heterojunction layer comprises an electron donor and an electron acceptor. Optionally, the bulk heterojunction layer consists of the electron donor and the electron acceptor.

Each of the anode and cathode may independently be a single conductive layer or may comprise a plurality of layers.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer. In some embodiments, a work function modification layer is disposed between the bulk heterojunction layer and the anode, and/or between the bulk heterojunction layer and the cathode.

The bulk heterojunction layer may be formed by any process including, without limitation, thermal evaporation and solution deposition methods.

Preferably, the bulk heterojunction layer is formed by depositing a formulation comprising the acceptor material and the electron donor material dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron acceptor, the electron donor and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

In the case where the organic photoresponsive device is an organic photodetector (OPD), it may be connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source.

In some embodiments, the light from the light source may or may not be changed before reaching the OPD. For example, the light may be reflected, filtered, down-converted or up-converted before it reaches the OPD.

At least one of the electron donor and electron acceptor of the bulk heterojunction layer is a material comprising a group of formula (I):

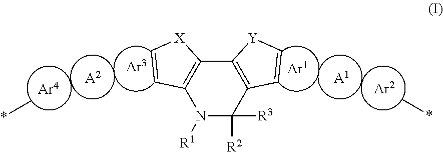

wherein:
X and Y are each independently selected from S, O or Se;
Ar¹, Ar², Ar³ and A⁴ are each independently an unsubstituted or a substituted benzene, an unsubstituted or a substituted 5- or 6-membered heteroaromatic group or are absent;
A¹ and A² are each independently an unsubstituted or a substituted benzene, an unsubstituted or a substituted 5- or 6-membered heteroaromatic group, a non-aromatic 6-membered ring having ring atoms selected from C, N, S and O or are absent;
R¹ is H or a substituent;
R² and R³ are each independently H or a substituent; and
* represents a point of attachment to a hydrogen or non-hydrogen substituent.

In a preferred embodiment, A¹ and A² are each independently a cyclohexane, wherein optionally one or more carbon atoms are replaced with S, NR¹ or O.

In some embodiments, the material comprising the group of formula (I) is a polymer comprising a repeat unit of formula (I). Preferably, the polymer is an electron donor of the bulk heterojunction layer.

In some embodiments, the material comprising the group of formula (I) is a non-polymeric compound containing at least one group of formula (I), optionally 1 or 2 groups of formula (I). Preferably, the non-polymeric compound is an electron acceptor of the bulk heterojunction layer and comprises at least one, optionally 1 or 2, electron donating groups of formula (I) and at least one electron-accepting group.

In preferred embodiments, the group of formula (I) has one of the following formulae:

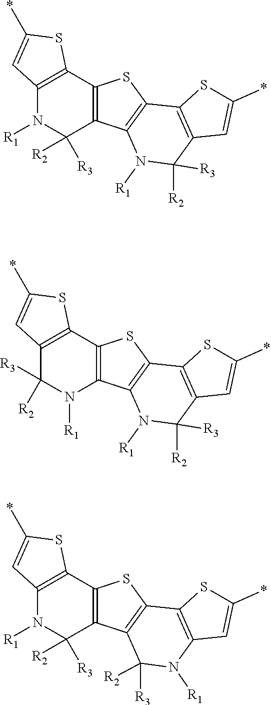

wherein R¹ is H or a substituent;

R² and R³ are each independently H or a substituent; and

* represents a point of attachment to a hydrogen or non-hydrogen group.

In some embodiments, the group of formula (I) is a group of formula (Ia) or formula (Ib):

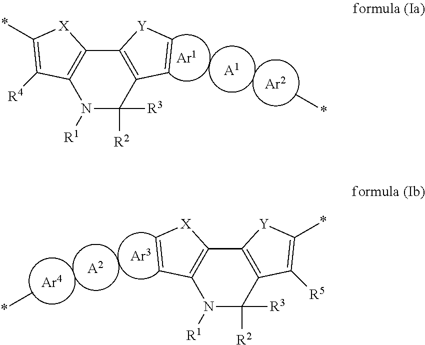

wherein:
X, Y, R¹, R² and R³ and * are as described previously for formula (I); and
R⁴ and R⁵ are each independently H or a substituent.

Optionally, R¹ is selected from: H; C₁₋₁₂ alkyl wherein one or more non-adjacent, non-terminal C atoms may be replaced with O, S, COO or CO and one or more H atoms of the alkyl may be replaced with F; and phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F.

Optionally, R² and R³ are each independently selected from H; C₁₋₂₀ alkyl wherein one or more non-adjacent, non-terminal C atoms may be replaced with O, S, COO or CO and one or more H atoms of the alkyl may be replaced with F; and an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO. R² and R³ may be linked to form a ring, e.g. a cycloalkyl ring or an aromatic or heteroaromatic ring, e.g. fluorene.

Optionally, R⁴ and R⁵ are each independently selected from H; F; and C₁₋₂₀ alkyl wherein one or more non-adjacent, non-terminal C atoms may be replaced with O, S, COO or CO and one or more H atoms of the alkyl may be replaced with F.

Ar¹-Ar⁴ are preferably each benzene or thiophene, each of which is optionally and independently unsubstituted or substituted with one or more substituents, optionally one or more substituents of formula R⁴.

In preferred embodiments, the group of formula (I) is a group of one of the following formulae:

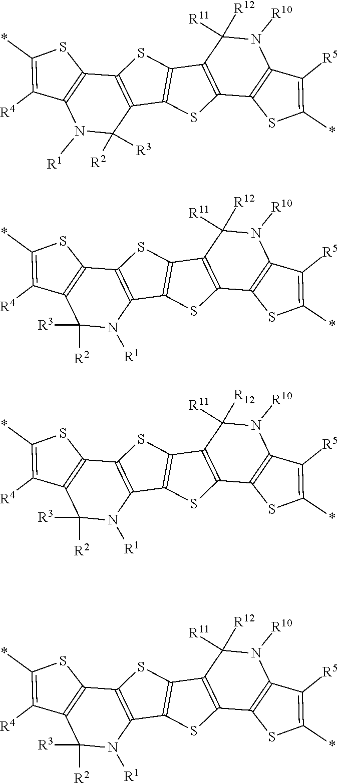

wherein:
* represents a point of attachment to a hydrogen or a non-hydrogen substituent;
R¹, R², R³, R⁴ and R⁵ are as defined previously for formulae Ia and Ib; and
R¹⁰, R¹¹ and R¹² are each independently H or a substituent, preferably a substituent selected from the group consisting of C₁₋₁₂ alkyl wherein one or more non-adjacent, non-terminal C atoms may be replaced with O, S, COO or CO and one or more H atoms of the alkyl may be replaced with F; and an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO.

In some embodiments, Ar¹, A¹, Ar², Ar³ A² and Ar⁴ of formula (I) are absent and the material has formula (Ic):

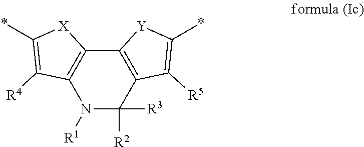

wherein:
X, Y, R¹, R² and R³ and * are as described previously for formula (I); and
R⁴ and R⁵ are as described previously form formulas (Ia) and (Ib).

In preferred embodiments, the group of formula (I) is selected from the following formulae:

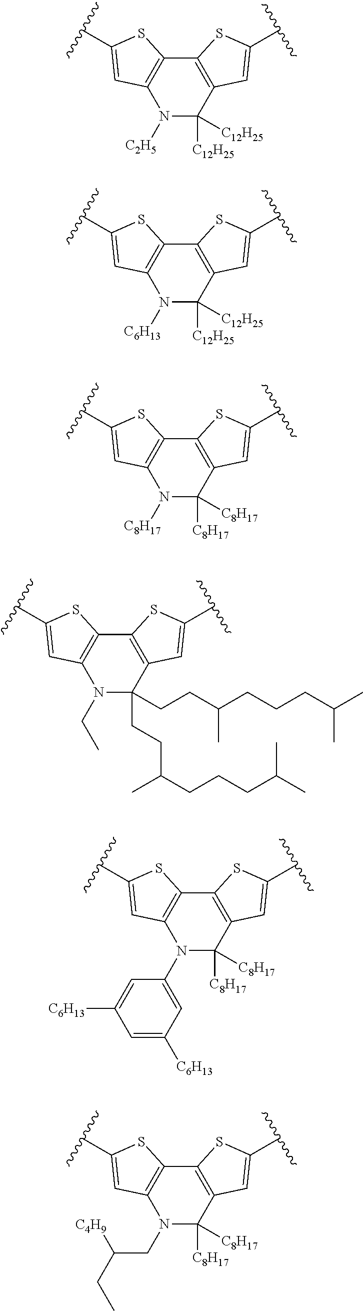

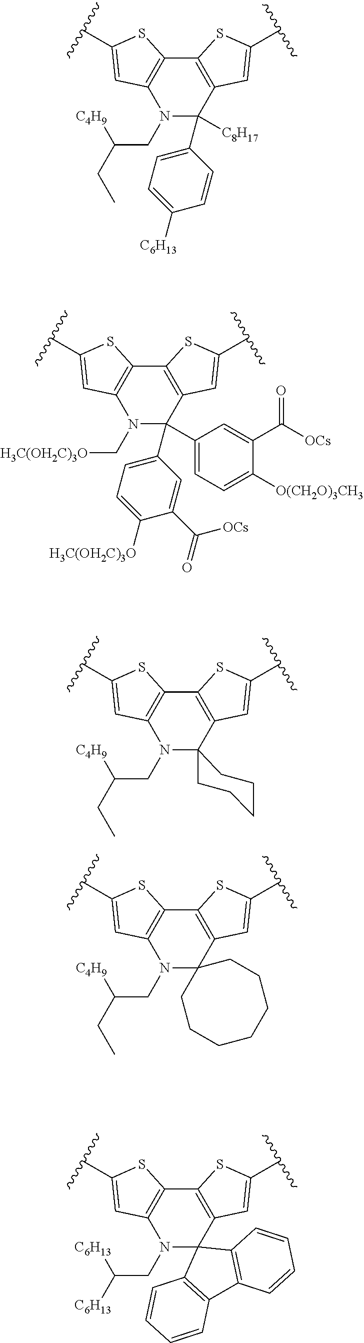

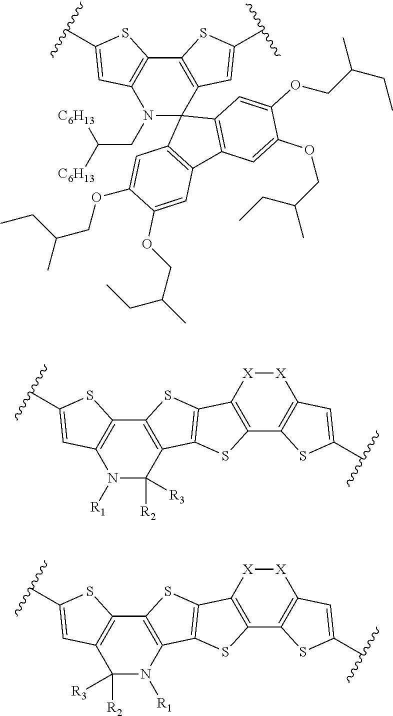

wherein one X is CR²R³ and the other X is O, S or NR¹.

In the case where the material comprising the group of formula (I) is a polymer, the polymer comprises a repeat unit of formula (Id):

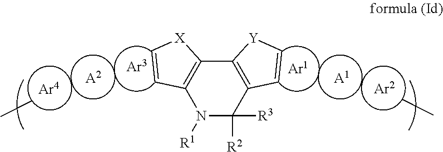

Optionally, the repeat unit of formula (Id) has formula (Ie), (If) or (Ig):

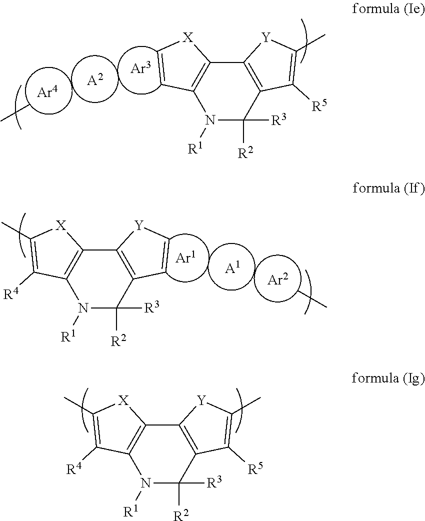

wherein X, Y, R¹ to R⁵, Ar¹ to Ar⁴, A¹ and A² are as previously defined.

The polymer is preferably a copolymer comprising electron-donating repeat units of formula (Id) and electron-accepting co-repeat units. Repeat units of formula (I) and the electron-accepting co-repeat units may together form a repeating structure in the polymer backbone of formula:

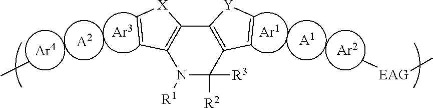

Optionally, each EAG repeat unit of the polymer (except any terminal EAG repeat unit) is adjacent to a repeat unit of formula (Id).

Optionally, each repeat unit of formula (Id) of the polymer, except any terminal repeat unit of formula (Id), is adjacent to an EAG repeat unit.

In the case where the material comprising a group of formula (I) is a non-polymeric compound, the compound preferably contains at least one electron accepting group (EAG) which may be directly or indirectly bound to the group of formula (I).

In a preferred embodiment, A¹ and A² are each independently a cyclohexane, wherein optionally one or more carbon atoms are replaced with S, NR¹ or O.

The, or each, EAG has a LUMO level that is deeper (i.e. further from vacuum) than EDG, preferably at least 1 eV deeper. The LUMO levels of EAG and EDG may be as determined by modelling the LUMO level of EAG-H or H-EAG-H with that of H-EDG-H, i.e. by replacing the bonds between EAG and EDG with bonds to a hydrogen atom. Modelling may be performed using Gaussian09 software available from Gaussian using Gaussian09 with B3LYP (functional) and LACVP* (Basis set).

Accordingly, in some embodiments, there is provided a material comprising a group of (Ih), formula (Ii), formula (Ij) or formula (Ik):

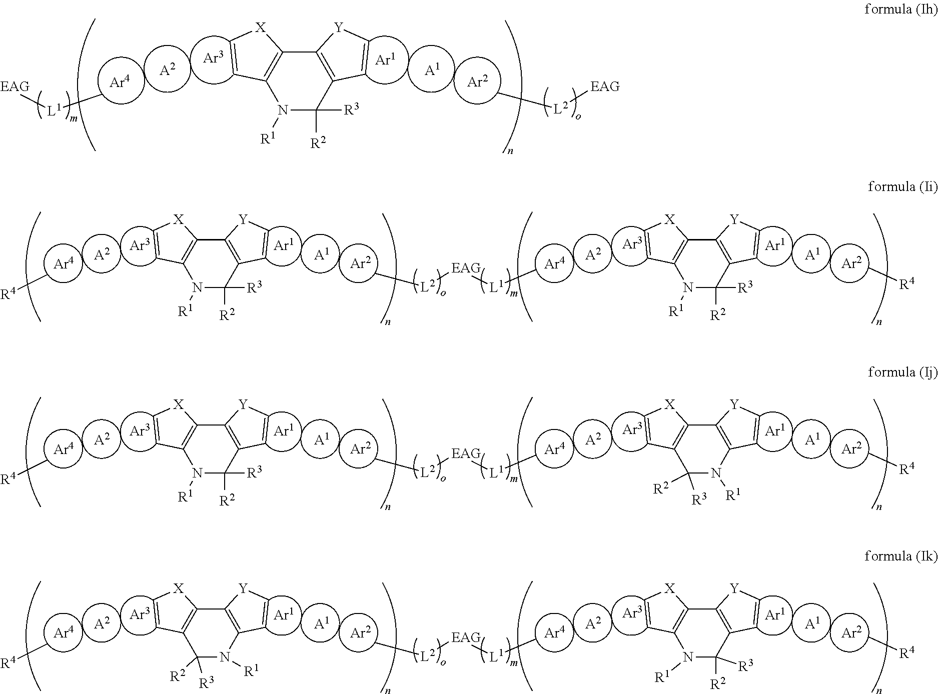

wherein:
n is an integer of 1 or more;
m and o are each independently 0 or an integer of 1 or more;
L¹ and L² each independently represent a bridging group when m and o are 1 or more or a direct bond when m and o are 0;
EAG represents an electron accepting group; and
X, Y, R¹ to R⁴, Ar¹ to Ar⁴, A¹ and A² are as previously defined.

Where the bridging groups L¹ and L² are present, L¹ and L² may each independently be a group of formula (II) or formula (III):

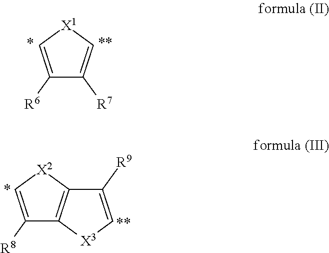

wherein:
X¹, X² and X³ are each independently S, O or Se;
* represents a point of attachment to Formula (Ih), Formula (Ii), Formula (Ij) or formula (Ik);
** represents a point of attachment to EAG; and
R⁶, R⁷, R⁸ and R⁹ are each independently H or a substituent.

Preferably, L¹ and L² are each independently selected from the following formulae:

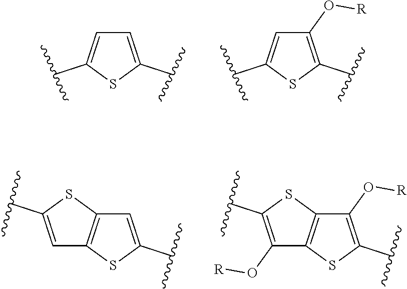

In the case where n is greater than 1, the groups of formula (I) may be linked in any orientation. For example, in the case where n=2, the two groups of formula (I) may be linked as:

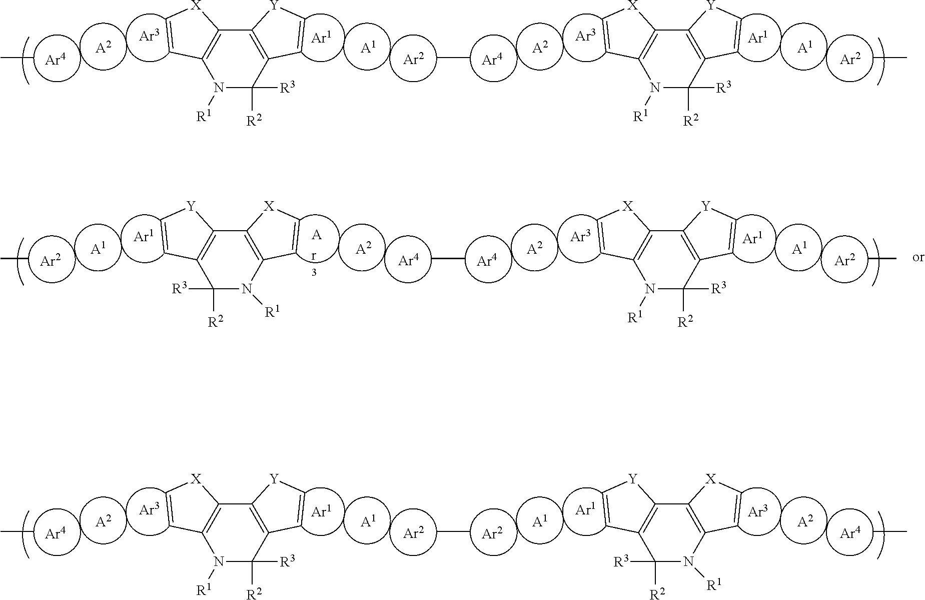

In the case where n is greater than 1, Ar¹-Ar⁴, A¹, A², R¹-R³, X and Y independently in each occurrence may be the same or different.

The monovalent EAGs of formula (Ih) may be the same or different, preferably the same. Optionally, each EAG of formula (Ih) is selected from formulae (III)-(XIV):

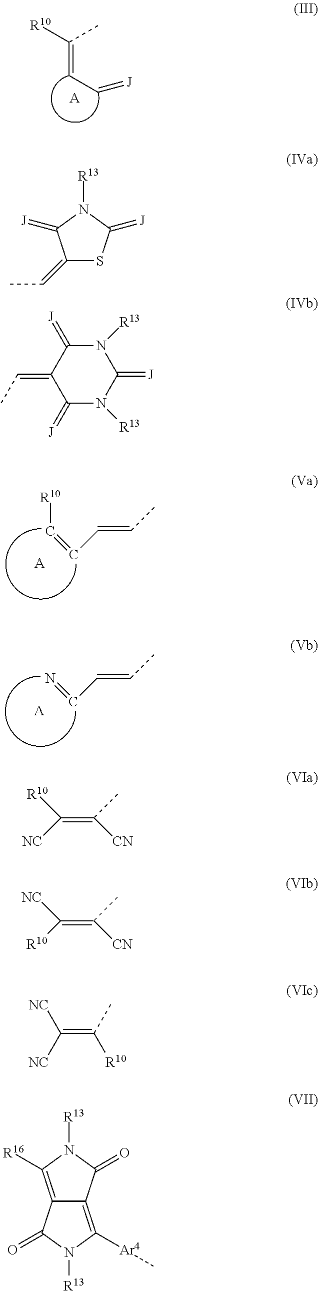

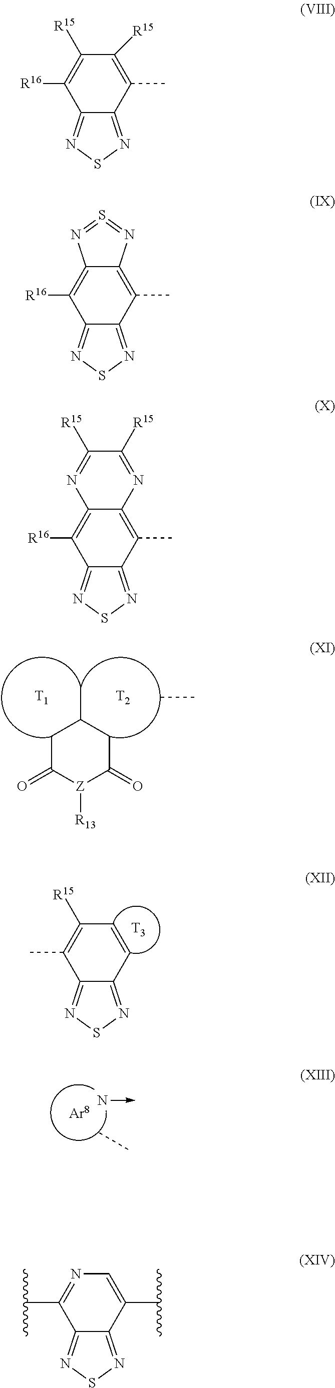

--- represents a bond to L¹, L² or a position denoted by * Formula (I)

A is a 5- or 6-membered ring which is unsubstituted or substituted with one or more substituents and which may be fused to one or more further rings.

R¹⁰ is H or a substituent, preferably a substituent selected from the group consisting of C₁₋₁₂ alkyl wherein one or more non-adjacent, non-terminal C atoms may be replaced with O, S, COO or CO and one or more H atoms of the alkyl may be replaced with F; and an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO.

Preferably, R¹⁰ is H.

J is O or S.

R¹³ in each occurrence is a substituent, optionally C₁₋₁₂ alkyl wherein one or more non-adjacent, non-terminal C atoms may be replaced with O, S, COO or CO and one or more H atoms of the alkyl may be replaced with F.

R¹⁵ in each occurrence is independently H; F; C₁₋₁₂ alkyl wherein one or more non-adjacent, non-terminal C atoms may be replaced with O, S, COO or CO and one or more H atoms of the alkyl may be replaced with F; or an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO.

R¹⁶ is a substituent, preferably a substituent selected from:

—(Ar³)_{w} wherein Ar³ in each occurrence is independently an unsubstituted or substituted aryl or heteroaryl group, preferably thiophene, and w is 1, 2 or 3;

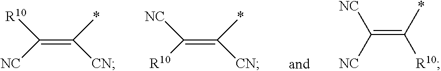

and

C₁₋₁₂ alkyl wherein one or more non-adjacent, non-terminal C atoms may be replaced with O, S, COO or CO and one or more H atoms of the alkyl may be replaced with F.

Ar⁴ is a 5-membered heteroaromatic group, preferably thiophene or furan, which is unsubstituted or substituted with one or more substituents.

Substituents of Ar³ and Ar⁴, where present, are optionally selected from C₁₋₁₂ alkyl wherein one or more non-adjacent, non-terminal C atoms may be replaced with O, S, COO or CO and one or more H atoms of the alkyl may be replaced with F.

Z¹ is N or P

T¹, T² and T³ each independently represent an aryl or a heteroaryl ring which may be fused to one or more further rings. Substituents of T¹, T² and T³, where present, are optionally selected from non-H groups of R¹⁵.

Ar⁸ is a fused heteroaromatic group which is unsubstituted or substituted with one or more non-H substituents R¹⁰.

A preferred group of formula (III) is formula (IIIa).

Preferably at least one, more preferably each, EAG is a group of formula (IIIa):

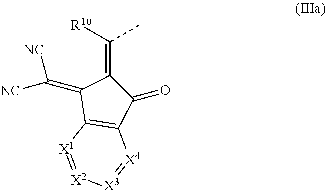

wherein:

R¹⁰ is as described above;

---- represents a linking position to L¹, L² or * of formula (I); and

each X¹-X⁴ is independently CR¹² or N wherein R¹² in each occurrence is H or a substituent selected from C₁₋₂₀ hydrocarbyl and an electron withdrawing group. Optionally, the electron withdrawing group is F, Cl, Br or CN.

The C₁₋₂₀ hydrocarbyl group R¹² may be selected from C₁₋₂₀ alkyl; unsubstituted phenyl; and phenyl substituted with one or more C₁₋₁₂ alkyl groups.

Exemplary compounds of formula (IVa) or (IVb) include:

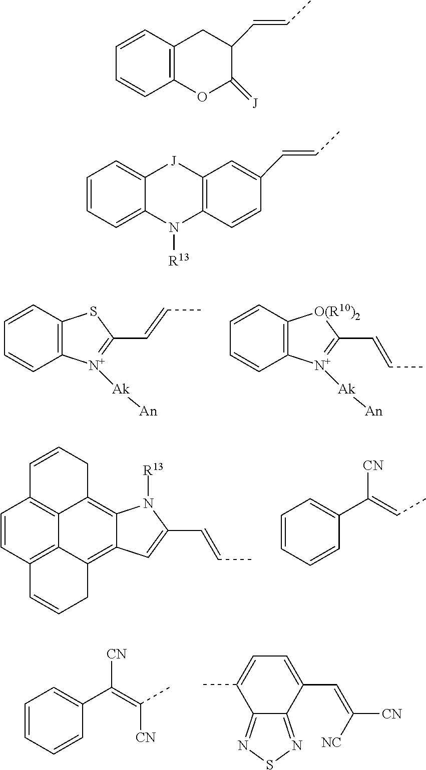

wherein Ak is a C₁₋₁₂ alkylene chain in which one or more C atoms may be replaced with O, S, CO or COO; An is an anion, optionally —SO₃⁻; and each benzene ring is independently unsubstituted or substituted with one or more substituents selected from substituents described with reference to R¹⁰.

Exemplary EAGs of formula (XI) are:

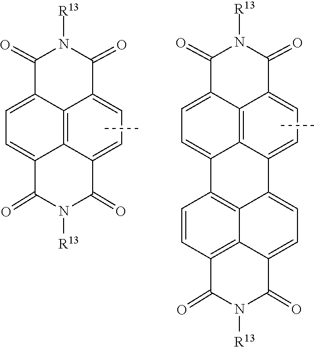

An exemplary EAG group of formula (XII) is:

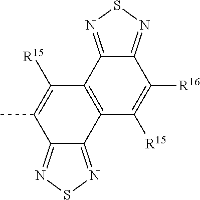

In the case where at least one EAG is a group of formula (XIII), the group of formula (I) is substituted with a group of formula —B(R¹⁴)₂ wherein R¹⁴ in each occurrence is a substituent, optionally a C₁₋₂₀ hydrocarbyl group; --- is bound to a position denoted by * in Formula (I); and → is a bond to the boron atom of —B(R¹⁴)₂.

Optionally, R¹⁴ is selected from C₁₋₁₂ alkyl; unsubstituted phenyl; and phenyl substituted with one or more C₁₋₁₂ alkyl groups.

The group of formula (I), the group of formula (XIII) and the B(R¹⁴)₂ substituent of formula (I) may be linked together to form a 5- or 6-membered ring.

In some embodiments, EAG of formula (XIII) is selected from formulae (XIIIa), (XIIIb) and (XIIIc):

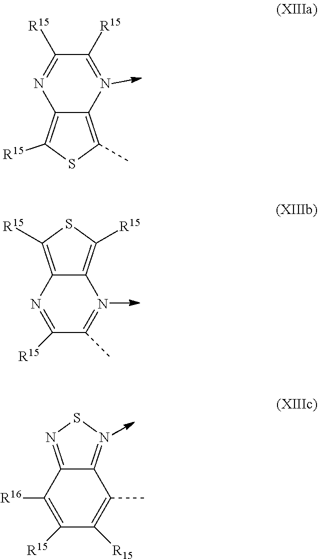

Divalent EAGs, for example of formula (Ii), (Ij) or (Ik) or EAG co-repeat units of a polymer comprising a repeat unit of formula (Id), are optionally selected from:
divalent analogues of formulae (VIII)-(X) wherein R¹⁶ is a bond to L¹, L² or * of formula (I); and
analogues (XIa) and (XIIa) of formulae (XI) and (XII), respectively:

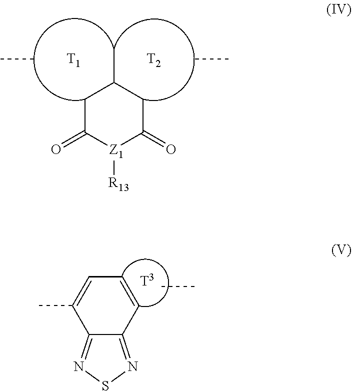

Preferable divalent EAGs, for example EAG repeat units of a polymer or EAG groups of a compound of formula (Ii), (Ij) or (Ik) are:

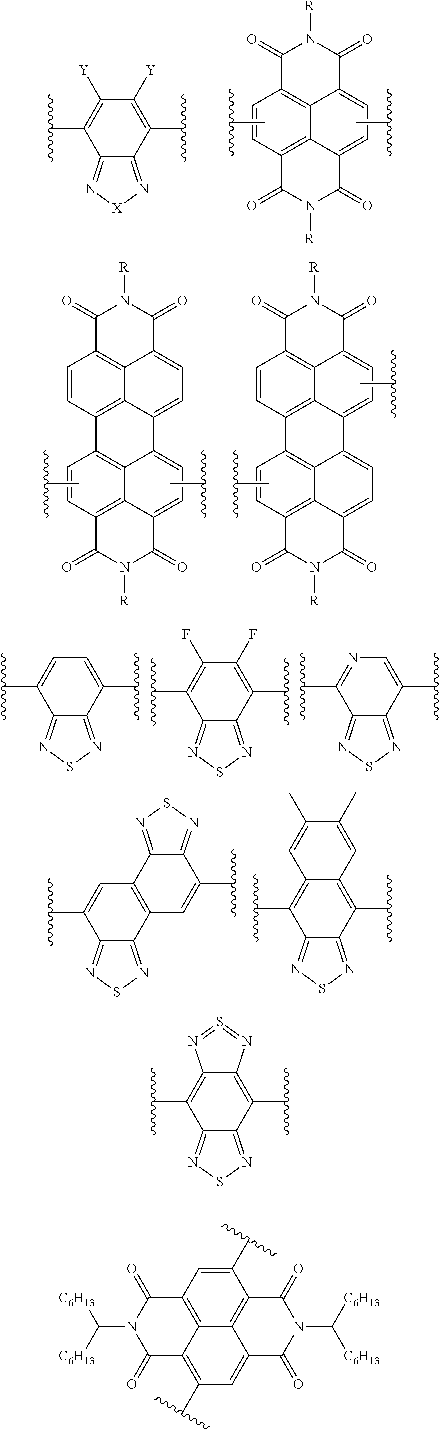

wherein Y is H or a substituent, e.g. a C₁₋₁₂ alkyl or F.

A photosensitive layer of an organic photoresponsive device as described herein may comprise or consist of a composition comprising an electron donor and an electron acceptor wherein at least one, optionally both, of the electron donor and electron acceptor is a material comprising a group of formula (I). The composition may contain only one electron acceptor and/or only one electron donor.

In the case where the composition comprises an electron donor comprising a group of formula (I), the composition may comprise one or more electron acceptors selected from non-fullerene acceptors, which may or may not comprise a group of formula (I), and fullerene acceptors.

Non-fullerene acceptors are described in, for example, Cheng et al, “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g. fluorinated derivatives thereof such as ITIC-4F and IEICO-4F.

Exemplary fullerene electron acceptor materials are C₆₀, C₇₀, C₇₆, C₇₈ and C₈₄ fullerenes or a derivative thereof including, without limitation, PCBM-type fullerene derivatives (including phenyl-C61-butyric acid methyl ester (C₆₀PCBM), TCBM-type fullerene derivatives (e.g. tolyl-C61-butyric acid methyl ester (C₆₀TCBM)), and ThCBM-type fullerene derivatives (e.g. thienyl-C61-butyric acid methyl ester (C₆₀ThCBM).

### EXAMPLES

Synthesis

A compound of formula (I) may be prepared according to the following reaction schemes:

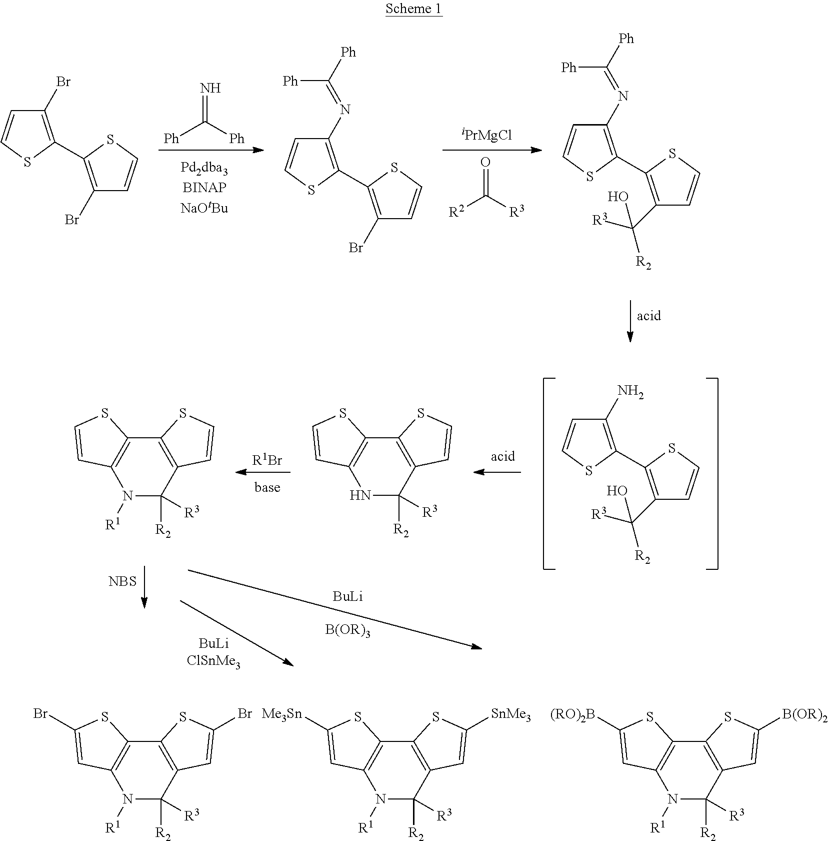

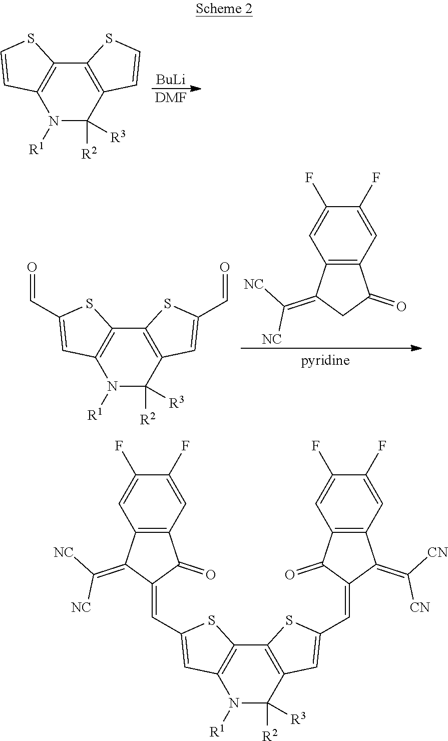

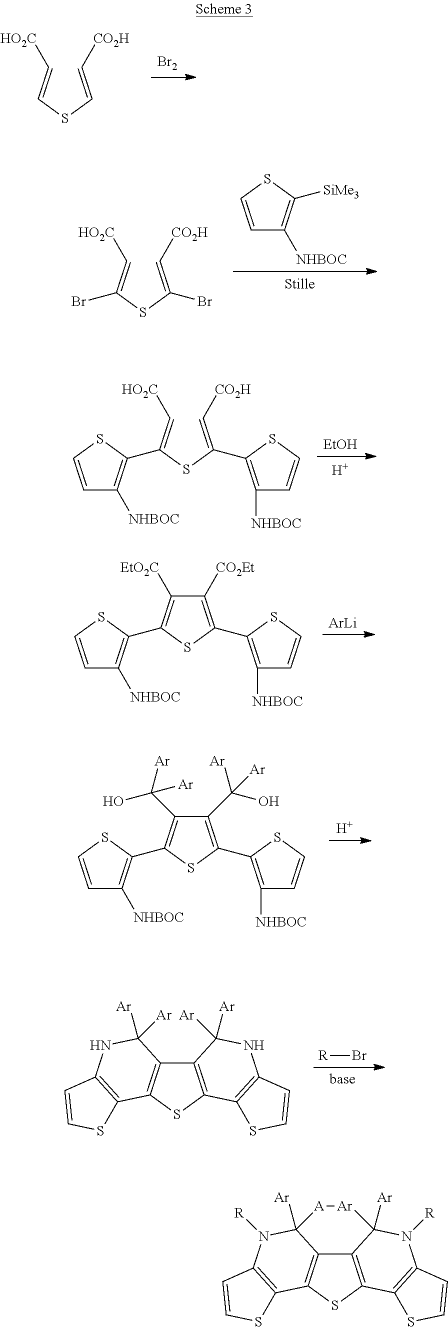

A compound of formula (Id) may be prepared according to the following reaction scheme:

wherein Ar is an aromatic group, optionally phenyl.

### Monomer Example 1

Monomer Example 1 was prepared according to the following reaction scheme:

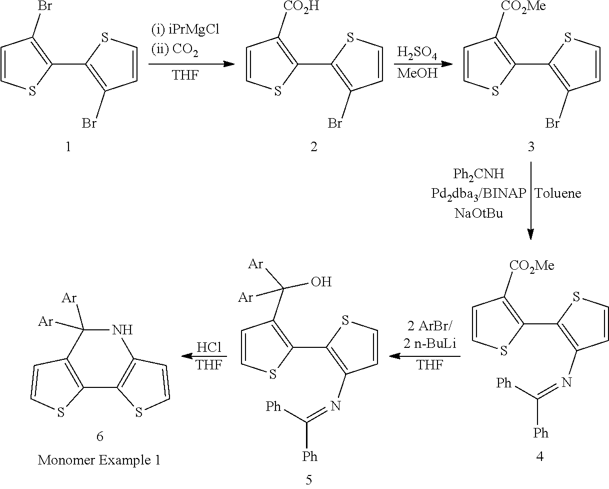

wherein Ar is

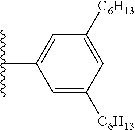

Preparation of 3

Isopropylmagnesium chloride (2 M in THF, 78.7 ml, 157.4 mmol, 1.7 equiv.) was added dropwise to a cold (0° C.) solution of 1 (30.0 g, 92.6 mmol) in anhydrous THF (250 ml) over twenty minutes. The solution was allowed to stir at 0° C. for one hour before warming to room temperature while stirring. The solution was briefly warmed to 45° C. before being allowed to cool back to room temperature and stirring at room temperature for 30 minutes. Consumption of starting material was confirmed by ¹H NMR spectroscopy of a 1 ml sample of the reaction mixture after quenching with methanol and removal of solvent. Under nitrogen flush, dry ice (˜30 g) was charged to a 500 ml 3-neck flask connected in series to a Dreschel bottle filled with activated 4 Å molecular sieves (followed by an empty Dreschel bottle to prevent suck-back) and a gas inlet tube was inserted into the cooled (0° C.) reaction mixture to allow a steady flow of dried CO₂/nitrogen to bubble through the stirred solution. A gradual colour change from pale orange to red was observed over one minute and the CO₂ purge was continued for a further 30 minutes before the CO₂ was disconnected, a dilute aqueous solution of HCl (50 ml) was carefully added and the mixture transferred to a separating funnel. Brine (50 ml) and water (50 ml) were added and the organic layer was separated. Solvents were removed under vacuum to afford a solid that was slurry washed with heptane/ethyl acetate (2:1 v/v, 150 ml), filtered and washed with heptane (50 ml), dried on the filter under suction to give 2 as an off-white free-flowing solid as ascertained by ¹H NMR spectroscopy.

Solid 2 was slurried in methanol (200 ml), concentrated sulfuric acid (1 ml) was added and the mixture heated to reflux overnight. The solution was allowed to cool to room temperature and solvent was removed under vacuum. The residue was dissolved in ethyl acetate (100 ml), washed with a dilute aqueous sodium carbonate solution (50 ml) and the organic layer separated, dried over anhydrous magnesium sulfate and reduced to dryness under vacuum. The product was loaded onto Celite and purified by column (340 g SNAP KP silica, EtOAc/heptane) to afford 3 as a colourless oil that solidifies on standing (14.05 g, 50%). HPLC purity 99.04%.

¹H NMR (CDCl₃, 298 K): δ 3.75 (s, 3H, H5); 7.06 (d, ³J_{HH}=4.5 Hz, 1H, H4); 7.38 (d, ³J_{HH}=4.5 Hz, 1H, H3); 7.40 (d, ³J_{HH}=4.5 Hz, 1H, H1); 7.54 (d, ³J_{HH}=4.5 Hz, 1H, H2) ppm.

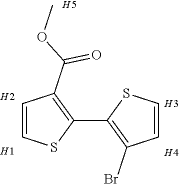

Preparation of 4

Anhydrous toluene (200 ml) was added to a mixture of 3 (13.75 g, 45.35 mmol), ±BINAP (219 mg, 0.351 mmol), benzophenone imine (7.61 ml, 45.35 mmol) and sodium tert-butoxide (6.10 g, 63.49 mmol) under nitrogen and the mixture degassed thoroughly for 30 minutes while stirring. Pd₂dba₃ (114 mg, 0.125 mmol) was added and the mixture heated to 80° C. overnight. The mixture was allowed to cool to room temperature and transferred to a separating funnel. Brine (50 ml) followed by water (50 ml) was added, the phases were thoroughly shaken and the organic phase separated, dried with anhydrous magnesium sulfate and filtered. Solvent was removed under vacuum and the oil was loaded onto Celite and purified by column (1500 g SNAP KP silica, EtOAc/heptane) to afford 4 as a yellow solid (10.81 g, 59%). HPLC purity 99.48%.

¹H NMR (CDCl₃, 298 K): δ 3.62 (s, 3H, H5); 6.44 (s, br, 1H, H4); 7.03 (d, ³J_{HH}=5.9 Hz, 2H, H6/10); 7.13 (d, ³J_{HH}=4.5 Hz, 1H, H1); 7.16 (d, ³J_{HH}=4.5 Hz, 1H, H3); 7.27 (t, ³J_{HH}=6.5 Hz, 2H, H7/9); 7.33 (t, ³J_{HH}=6.3 Hz, ¹H, H8); 7.34 (d, ³J_{HH}=4.5 Hz, ¹H, H2); 7.37 (t, ³J_{HH}=6.3 Hz, 2H, H12/14); 7.45 (t, ³J_{HH}=6.2 Hz, 1H, H13); 7.70 (d, ³J_{HH}=6.0 Hz, 2H, H11/15) ppm.

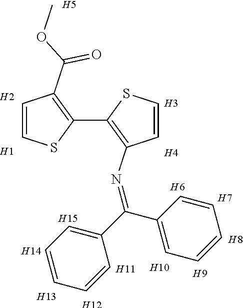

Preparation of 5

n-Butyllithium (2.5 M in hexanes, 19.8 ml, 49.6 mmol) was added dropwise over 10 minutes to a cold (−78° C.) degassed solution of 1-bromo-3,5-dihexylbenzene (16.12 g, 49.6 mmol) in anhydrous THF (250 ml) under nitrogen. The yellow solution was stirred at −78° C. for 2 hours and 4 was added carefully to the stirring mixture, which was allowed to warm to room temperature and stirred for a further 16 hours. The brown solution was cooled to 0° C. and methanol (˜50 ml) was added. Solvents were removed under vacuum and the oily residue was extracted with ethyl acetate (100 ml), washed with brine (50 ml) and water (50 ml) and dried over anhydrous magnesium sulfate. Solvent was removed under vacuum and the oil was loaded onto Celite and purified by column (120 g Sfår C18 D silica, MeCN/THF (BHT-free)) to afford a yellow-brown oil (15.25 g). HPLC purity 90.56%. This material was re-purified by column (950 g SNAP KP-C18 HS, MeCN/THF (BHT-free)) to give 5 as a yellow oil (12.99 g, 66%). HPLC purity 95.11%.

¹H NMR (CDCl₃, 298 K): δ 0.83 (t, ³J_{HH}=5.8 Hz, 12H, H12); 1.24 (m, 24H, H9/10/11); 1.51 (m, 8H, H8); 2.45 (m, 8H, H7); 5.67 (d, ³J_{HH}=4.5 Hz, 1H, H2); 6.47 (d, ³J_{HH}=4.5 Hz, 1H, H1); 6.66 (d, ³J_{HH}=4.5 Hz, 1H, H3); 6.72 (s, 2H, H6); 7.06 (d, ³J_{HH}=5.9 Hz, 2H, H13/17); 7.11 (s, 4H, H5); 7.12 (d, 1H, H4); 7.29 (t, ³J_{HH}=6.0 Hz, 2H, H14/16); 7.34 (t, ³J_{HH}=6.2 Hz, 1H, H15); 7.42 (t, ³J_{HH}=6.2 Hz, 2H, H18/22); 7.49 (t, ³J_{HH}=6.2 Hz, 1H, H20); 7.70 (d, ³J_{HH}=6.1 Hz, 2H, H19/21) ppm.

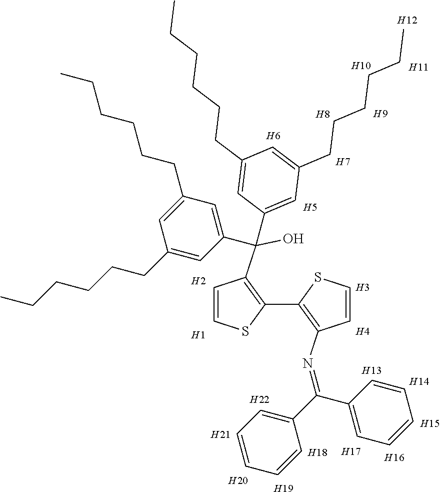

Preparation of 6

Concentrated HCl (1.6 ml, 18.2 mmol) was added to a cold (0° C.) solution of 5 in THF (˜50 ml) and stirred for one hour, monitoring consumption of starting material by ¹H NMR spectroscopy. Solvent was removed under vacuum and the residue was extracted with DCM (50 ml), washed with water (2×20 ml) and dried over anhydrous magnesium sulfate. Solvent was removed under vacuum and the oil purified by column (950 g SNAP KP-C18 HS, MeCN/acetone) to afford 6 as a yellow oil that crystallises on standing over 1 week (10.56 g, 85%). HPLC purity 96.01%.

¹H NMR (CDCl₃, 298 K): δ 0.86 (t, ³J_{HH}=7.0 Hz, 12H, H12); 1.25 (m, 24H, H9/10/11); 1.51 (m, 8H, H8); 2.49 (t, ³J_{HH}=7.8 Hz, 8H, H7); 6.46 (d, ³J_{HH}=4.9 Hz, 1H, H2); 6.55 (d, ³J_{HH}=5.0 Hz, 1H, H4); 6.86 (s, 2H, H6); 6.87 (s, 4H, H5); 6.89 (d, ³J_{HH}=4.9 Hz, 1H, H1); 6.93 (d, ³J_{HH}=5.1 Hz, 1H, H3) ppm.

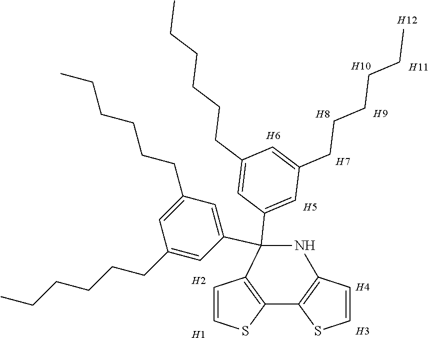

Synthesis of Polymer Example 1

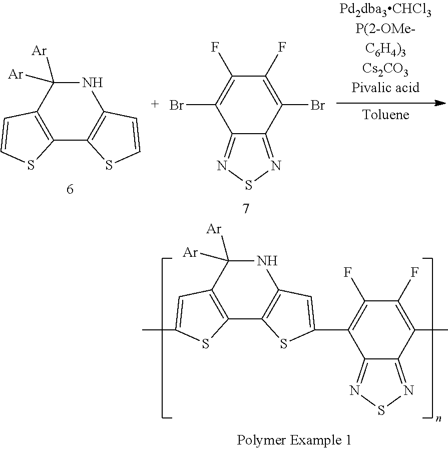

The following were charged to a 100 ml 3-neck round bottom flask fitted with thermometer and condenser containing a magnetic stir bar: 6 (1.0657 g, 1.500 mmol, 1 equiv), 7 (0.4949 g, 1.500 mmol, 1 equiv), Pd₂dba₃.CHCl₃ (7.76 mg, 7.5 μmol, 0.5 mol %), tris(2-methoxyphenyl)phosphine (10.57 mg, 30.0 mol, 2.0 mol %), anhydrous caesium carbonate (1.4661 g, 4.500 mmol, 3 equiv) and pivalic acid (153.19 mg, 1.500 mmol, 1 equiv). The flask was flushed with nitrogen for 20 minutes. 25 ml dry toluene was added via cannula under nitrogen and the mixture was slowly stirred while degassing through an inlet tube for 20 minutes. The mixture was heated to 100° C. for 48 hours, allowed to cool and further portions of Pd₂dba₃.CHCl₃ (7.76 mg, 7.5 mol, 0.5 mol %) and tris(2-methoxyphenyl)phosphine (10.57 mg, 30.0 μmol, 2.0 mol %) were added before heating was resumed at 100° C. for 10 days. Analysis of the reaction mixture by LCMS revealed the reaction had stalled so solvent was removed, the residue was extracted with EtOAc (100 ml), washed with water, separated, dried over anhydrous magnesium sulfate and filtered through Celite, silica and Florisil. Solvent was removed and to the dry residue was added Pd₂dba₃.CHCl₃ (15.52 mg, 15.0 mol, 1.0 mol %), tris(2-methoxyphenyl)phosphine (21.14 mg, 60.0 μmol, 4.0 mol %), anhydrous caesium carbonate (1.4661 g, 4.500 mmol, 3 equiv) and pivalic acid (153.19 mg, 1.500 mmol, 1 equiv). The flask was flushed with nitrogen for 20 minutes. 25 ml dry toluene was added via cannula under nitrogen and the mixture was slowly stirred while degassing through an inlet tube for 20 minutes. The mixture was heated to 100° C. under nitrogen for another 7 days before the mixture was allowed to cool and solvent was removed. The dark blue-green residue was extracted with mesitylene (50 ml), washed with water (2×50 ml), washed twice with sodium diethyldithiocarbamate trihydrate solutionhe (each wash contained 2.5 g in 50 ml water) at 65° C. for 30 mins, washed with 50 ml 10% AcOH solution at 65° C. for 15 mins and washed twice with 50 ml water at 65° C. for 15 mins. The solution was allowed to cool and added slowly into 300 ml stirring MeOH to form a fine precipitate, which was filtered, washed with MeOH (2×30 ml) and dried on the filter to give 760 mg solid. This was dissolved in 40 ml mesitylene at 50° C., the solution was filtered, reduced in volume to 2-3 ml and added dropwise into 300 ml stirring MeOH to precipitate a fibrous dark material. The solid was isolated by filtration, washed with MeOH (2×30 ml) and dried under vacuum at 50° C. for 48 hours to afford 500 mg of Polymer Example 1 (39% yield). Mw by Rapid GPC: 15,000 g mol⁻¹.

Square Wave Voltammetry

HOMO and LUMO levels of Polymer Example 1 were measured by square wave voltammetry (SWV) in solution and the values are provided in Table 1 with values for Comparative Polymer 1. As shown in Table 1 the HOMO-LUMO bandgap of Polymer Example 1 is significantly smaller than that of Comparative Polymer 1.

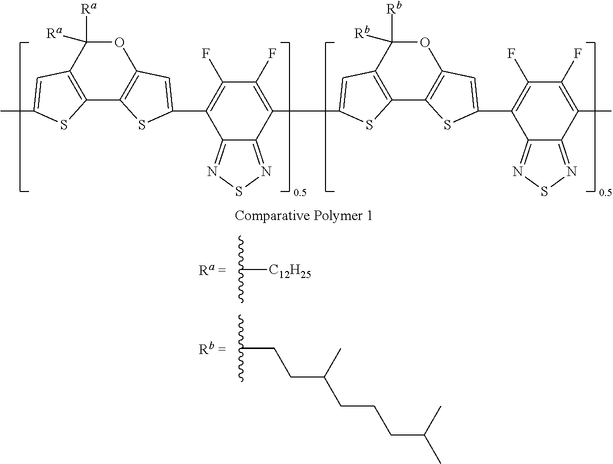

TABLE 1

| Polymer | HOMO (eV) | LUMO (eV) | Band Gap (nm) |
|---|---|---|---|
| Comparative Polymer 1 | −5.33 | −3.17 | 574 |
| Polymer Example 1 | −4.96 | −3.19 | 713 |

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO and LUMO energy levels by SWV comprises a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml).

LUMO=4.8-E ferrocene (peak to peak average)−E reduction of sample (peak maximum).

HOMO=4.8-E ferrocene (peak to peak average)+E oxidation of sample (peak maximum).

A typical SWV experiment runs at 15 Hz frequency; 25 mV amplitude and 0.004 V increment steps. Results are calculated from 3 samples for both the HOMO and LUMO data.

Absorption Data

Absorption spectra for Polymer Example 1 and Comparative Polymer 1 are shown in FIG. 2. Spectra were recorded using a Perkin Elmer Cary 5000 UV-vis absorption spectrometer of a film of the polymer cast onto a quartz substrate by spin-coating a 15 mg/ml solution of the polymer in toluene using spinner SCS Version 1.6; Model 6712D, with a spinner program of 500 rpm for 45 seconds followed by 4000 rpm for 5 seconds.

As shown in FIG. 2, Polymer Example 1 has a peak of 752 nm and absorption of Polymer Example 1 is stronger at wavelengths above about 850 nm.

Modelling Data

HOMO and LUMO levels of the following compounds were modelled and results are set out in Table 2. Quantum chemical modelling was performed using Gaussian09 software available from Gaussian using Gaussian09 with B3LYP (functional) and LACVP* (Basis set).

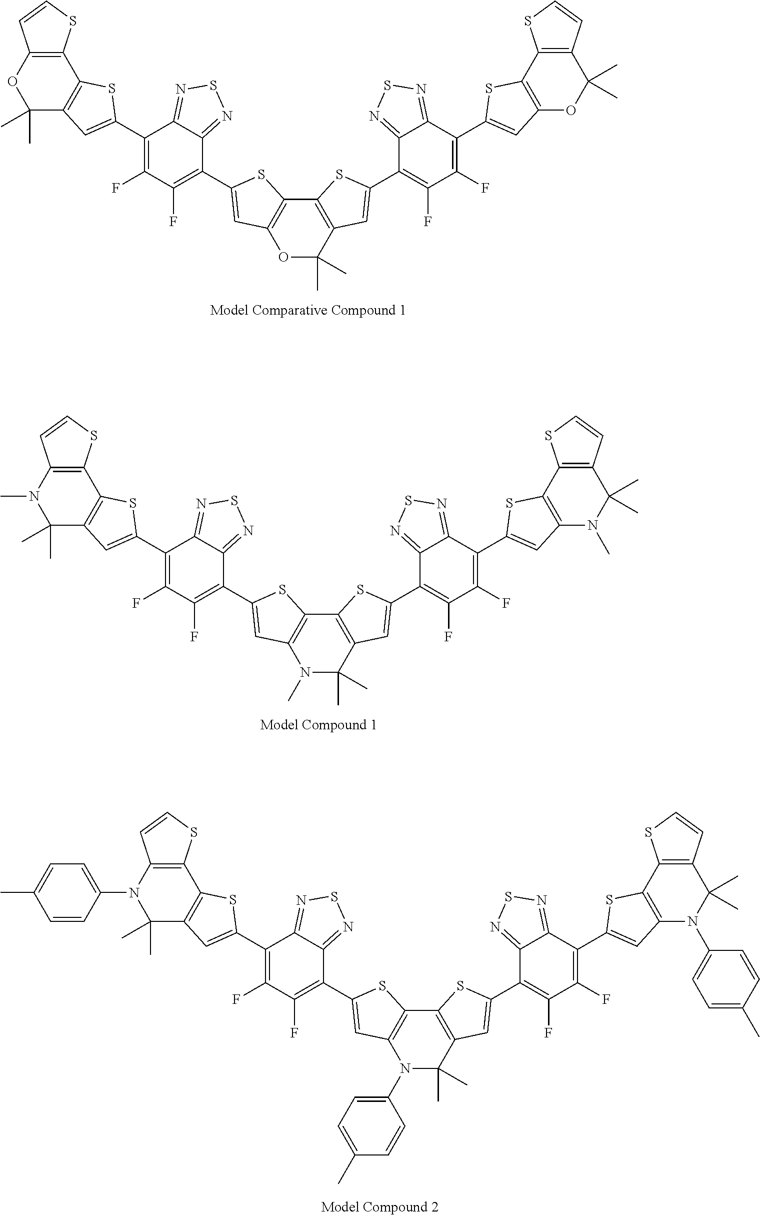

TABLE 2

| | HOMO | LUMO | Band gap |
|---|---|---|---|
| Compound | (eV) | (eV) | (eV) |
| Model Comparative | −4.654 | −2.923 | 1.731 |
| Compound | | | |
| Model Compound 1 | −4.286 | −2.744 | 1.542 |
| Model Compound 2 | −4.216 | −2.660 | 1.556 |

The donor group of Model Compound Example 1 comprising an N-substituted methyl group has a smaller band gap than the Model Comparative Compound with a different central fused group, indicating that Model Compound 1 is capable of absorbing light at longer wavelengths than the Model Comparative Compound.

Model Compound 2, in which the donor group comprises an N-substituted p-tolyl group, has an even smaller band gap than both the Model Comparative Compound and Model Compound 1 and is capable of absorbing light at longer wavelengths than the Model Comparative Compound.

1. A material comprising an electron-accepting group (EAG) and an electron-donating group (EDG) wherein EDG is a group of formula (I): 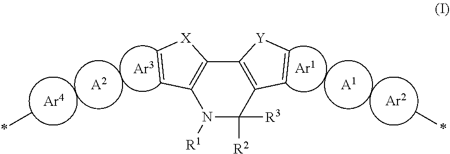
wherein:
X and Y are each independently selected from S, O or Se;
Ar¹, Ar², Ar³ and Ar⁴ are each independently an unsubstituted or a substituted benzene, an unsubstituted or a substituted 5- or 6-membered heteroaromatic group or are absent;
A¹ and A² are each independently an unsubstituted or a substituted benzene, an unsubstituted or a substituted 5- or 6-membered heteroaromatic group, a non-aromatic 6-membered ring having ring atoms selected from C, N, S and O or are absent;
R¹ is H or a substituent;
R² and R³ are each independently H or a substituent; and
----- represents a point of attachment to a hydrogen or non-hydrogen group.

2. A material as claimed in claim 1, wherein the group of formula I has formula (Ia) or formula (Ib): 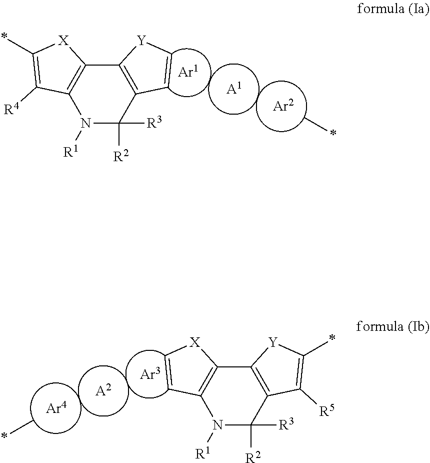
wherein:
X, Y, R¹, R² and R³ and ----- are as defined in claim 1; and
R⁴ and R⁵ are each independently H or a substituent.

3. A material as claimed in claim 1, wherein Ar¹, A¹, Ar², Ar³ A² and Ar⁴ are absent and the group of formula (I) has formula (Ic): 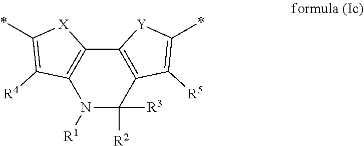
wherein:
X, Y, R¹, R², R³, R⁴, R⁵ and * are as defined in claims 1 and 2.

4. A material as claimed in claim 1, wherein the material is a polymer-comprising, an electron-accepting repeat unit and an electron-donating repeat unit and wherein the electron-accepting repeat unit is a repeat unit of formula (Id):

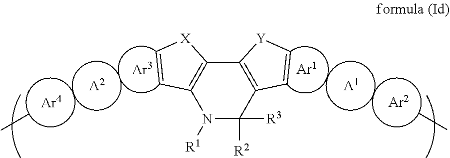

5. A material as claimed in claim 4, wherein the repeat unit of formula (Id) is selected from repeat units of formulae (Ie), (If) and (Ig): 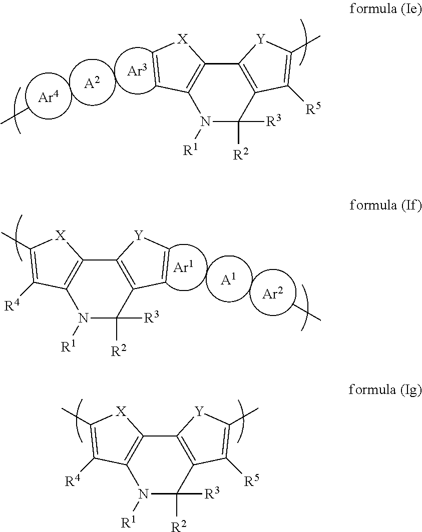
wherein X, Y, R¹ to R⁵, Ar¹ to Ar⁴, A¹ and A² are as previously defined in claims 1 and 2.

6. (canceled)

7. A material as claimed in claim 1, wherein the material comprising the group of formula (I) is selected from formulae (Ih), (Ii), (Ij) and (Ik): 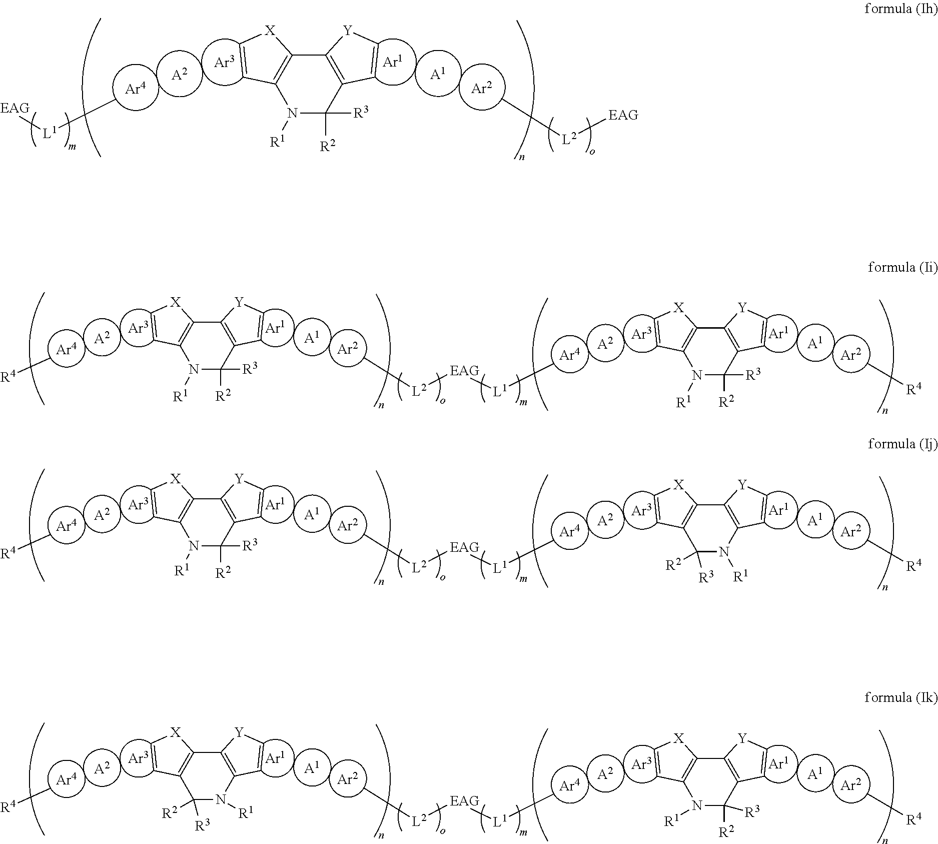
wherein:
n is an integer of 1 or more;
m and o are each independently 0 or an integer of 1 or more;
L¹ and L² each independently represent a bridging group when m and o are 1 or more or a direct bond when m and o are 0;
EAG represents an electron accepting group; and
X, Y, R¹ to R⁴, Ar¹ to Ar⁴, A¹ and A² are as previously defined in claims 1 and 2.

8. A material as claimed in claim 7, wherein L¹ and L² are each independently a group of formula (II) or formula (III): 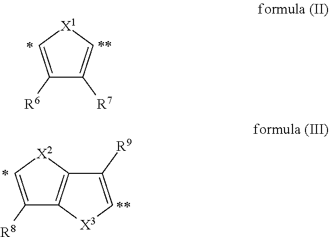
wherein:
X¹, X² and X³ are each independently S, O or Se;
* represents a point of attachment to Formula (Ih), Formula (Ii) or Formula (Ij);
** represents a point of attachment to EAG; and
R⁶, R⁷, R⁸ and R⁹ are each independently H or a substituent.

9. A material according to claim 7 wherein each EAG is a group of formula (VIa): 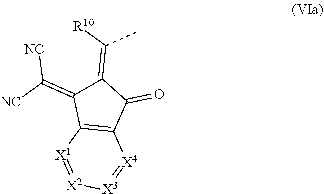
wherein:
R¹⁰ in each occurrence is H or a substituent selected from the group consisting of: C₁₋₁₂ alkyl wherein one or more non-adjacent, non-terminal C atoms may be replaced with O, S, COO or CO and one or more H atoms of the alkyl may be replaced with F; and an aromatic group Ar² which is unsubstituted or substituted with one or more substituents selected from F and C₁₋₁₂ alkyl wherein one or more non-adjacent, non-terminal C atoms may be replaced with O, S, COO or CO;
---- represents a linking position to Formula (Ih), Formula (Ii), Formula (Ij), L¹ or L²; and each X¹-X⁴ is independently CR¹² or N wherein R¹² in each occurrence is H or a substituent selected from C₁₋₂₀ hydrocarbyl and an electron withdrawing group.

10. The material according to claim 9 wherein at least one R¹² is an electron-withdrawing group selected from F, Br, Cl and CN.

11. A composition comprising an electron donor and an electron acceptor wherein at least one of the electron donor and electron acceptor is a material according to claim 1.

12. A photoresponsive device comprising an anode, a cathode and a photosensitive layer disposed between the anode and the cathode, wherein the photosensitive layer comprises a material according to claim 1.

13. A photoresponsive device as claimed in claim 12, wherein the photoresponsive device is an organic photodetector.

14. A method of forming an organic photoresponsive device according to claim 12 comprising formation of the photosensitive organic layer over one of the anode and cathode and formation of the other of the anode and cathode over the photosensitive organic layer.

15. A method according to claim 14 wherein formation of the photosensitive organic layer comprises deposition of a formulation comprising a composition comprising an electron donor and an electron acceptor, wherein at least one of the electron donor and electron acceptor is the material, dissolved or dispersed in one or more solvents.

16. A photosensor comprising a light source and a photoresponsive device as claimed in claim 12, wherein the photosensor is configured to detect light emitted from a light source.

17. A photosensor according to claim 16, wherein the light source emits light having a peak wavelength greater than 750 nm.

18. A photosensor according to either claim 16 configured to receive a sample in a light path between the organic photodetector and the light source.

19. A method of determining the presence and/or concentration of a target material in a sample, the method comprising illuminating the sample and measuring a response of a photoresponsive device as claimed in claim 12

20. A formulation comprising a material as claimed in claim 1 or a composition according to claim 11 dissolved or dispersed in one or more solvents.
